(19) United States
(12) Patent Application Publication
(10) Pub. No.: US 2013/0142723 A1
(43) Pub. Date: Jun. 6, 2013

(54) BIOMASS GASIFICATION SYSTEMS HAVING CONTROLLABLE FLUID INJECTORS

(71) Applicant: Satya Dileep Dara, Bangalore (IN)

(72) Inventor: Satya Dileep Dara, Bangalore (IN)

(73) Assignee: General Electric Company, Schenectady, NY (US)

(21) Appl. No.: 13/312,280

(22) Filed: Dec. 6, 2011

(51) Int. Cl. C01C 1/02; C07C 31/04; C10J 3/46; C10J 3/72; G05D 9/00

(52) U.S. Cl. 423/352; 48/87; 422/106; 48/197 R; 568/840

(57) ABSTRACT

Biomass gasification systems including a reactor adapted to gasify a biomass feedstock to thermally convert the biomass feedstock into producer gas are provided. The reactor includes an enclosure disposed about a biomass gasification chamber. The enclosure includes an inlet, an outlet, and side walls disposed between the inlet and the outlet. The reactor also includes a plurality of fluid injectors disposed along a length of the side walls and adapted to inject fluid into the gasification chamber. The biomass gasification system also includes a control system communicatively coupled to the plurality of fluid injectors and adapted to independently control each fluid injector of the plurality of fluid injectors to independently control a flow of fluid through each fluid injector.

### BACKGROUND OF THE INVENTION

The subject matter disclosed herein relates generally to gasification systems, and more particularly, to biomass gasification systems having controllable injection nozzles.

Gasification is a process that has become ubiquitous in various industries and applications for conversion of a lower, less readily usable type of fuel into a higher form of fuel. For example, biomass gasification systems are utilized in a variety of types of power plants to pyrolytically convert biomass via heating with air or oxygen to generate producer gas composed of gases such as carbon monoxide, carbon dioxide, hydrogen, methane, and nitrogen. This producer gas is then utilized to make methanol, ammonia, and diesel fuel through known commercial catalytic processes. In such a way, various forms of organic waste, such as wood, coconut shell fibers, alcohol fuels, and so forth, may be gasified for use in the production of electricity for a variety of downstream applications.

Unfortunately, many current biomass gasification systems generate producer gas with high levels of undesirable particulates, such as tar. Accordingly, prior to use in a power generation system, the producer gas needs to be cleaned to generate a gas mixture with the desired composition. The incorporation of cleaning components, such as scrubbers and filters, for removing these undesirable particulates can add cost and complexity to the biomass gasification system, thus reducing its efficiency. Accordingly, there exists a need for biomass gasification systems capable of generating relatively clean producer gas while overcoming these drawbacks.

### BRIEF DESCRIPTION OF THE INVENTION

Certain embodiments commensurate in scope with the originally claimed invention are summarized below. These embodiments are not intended to limit the scope of the claimed invention, but rather these embodiments are intended only to provide a brief summary of possible forms of the invention. Indeed, the invention may encompass a variety of forms that may be similar to or different from the embodiments set forth below.

In a first embodiment, a biomass gasification system includes a reactor adapted to gasify a biomass feedstock to thermally convert the biomass feedstock into producer gas are provide. The reactor includes an enclosure disposed about a biomass gasification chamber. The enclosure includes an inlet, an outlet, and a pair of side walls disposed between the inlet and the outlet. The reactor also includes a plurality of fluid injectors disposed along a length of the side walls and adapted to inject fluid into the gasification chamber. The biomass gasification system also includes a control system communicatively coupled to the plurality of fluid injectors and adapted to independently control each fluid injector of the plurality of fluid injectors to independently control a flow of fluid through each fluid injector.

In a second embodiment, a biomass gasification system includes a reactor adapted to gasify a biomass feedstock in a biomass gasification chamber to thermally convert the biomass feedstock into producer gas. A plurality of nozzles are disposed along a length of the reactor and adapted to inject air and/or oxygen into the biomass gasification chamber. The biomass gasification system also includes a control system adapted to determine an approximate location of a combustion zone in the gasification chamber and to selectively deactivate each nozzle of the plurality of nozzles not capable of injecting air and/or oxygen into the combustion zone.

In a third embodiment, a method includes the step of receiving data corresponding to an operational parameter of a biomass gasification process or a power generation system that receives producer gas from the biomass gasification process. The method also includes activating, based on the received data, a subset of a plurality of air and/or oxygen injectors disposed along a length of a biomass gasifier and about a biomass gasification chamber. The method further includes controlling an air delivery system to supply the activated subset of the plurality of air and/or oxygen injectors with air and/or oxygen for injection into the biomass gasification chamber.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an embodiment of a biomass gasification system including a biomass gasifier having a plurality of controllable fluid injectors;

FIG. 2 is a diagram of an embodiment of a biomass gasifier having a plurality of fluid injectors and an embodiment of an air delivery system capable of selectively delivering air to the air injection nozzles;

FIG. 3 illustrates an embodiment of a method for selectively activating a plurality of controllable fluid injectors based on an operational parameter of a biomass gasification process;

FIG. 4 illustrates an embodiment of a method for selectively activating a plurality of controllable fluid injectors based on a type of feedstock utilized in a biomass gasification process;

FIG. 5 illustrates an embodiment of a method for selectively activating a plurality of controllable fluid injectors based on a detected tar content of a producer gas generated in a biomass gasification process; and

FIG. 6 illustrates an embodiment of a method for selectively activating a plurality of controllable fluid injectors based on an operational parameter of an engine located in a power generation system downstream of a biomass gasification chamber.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements.

As described below, provided herein are fuel conversion systems including thermal conversion devices having a plurality of controllable fluid injectors that inject air and/or oxygen into a chamber of the thermal conversion device. These controllable fluid injectors may be disposed in a variety of systems and devices, such as various types of gasification systems typically found in industrial equipment, power plants, or other applications. For example, in certain embodiments, the controllable fluid injectors may be incorporated into a reactor, such as a biomass gasifier of a biomass gasification system, to inject air and/or oxygen into a gasification chamber. That is, the controllable fluid injectors may be included in a biomass gasification system capable of converting biomass into a higher, potentially more useful, type of fuel. For instance, the biomass gasification system may gasify biomass, for example, pyrolytically via heating with air or oxygen, to generate producer gas having varying concentrations of gases such as carbon monoxide, carbon dioxide, hydrogen, methane, and nitrogen, as well as particulate matter of various sizes.

The controllable fluid injectors described herein facilitate this conversion of biomass into producer gas by injecting air and/or oxygen into a combustion zone of a biomass gasification chamber. The controllability of the fluid injectors may enable the selective activation and deactivation of subsets of the plurality of fluid injectors. The embodiments described herein may offer distinct advantages over traditional biomass gasification systems that typically do not include controllable fluid injectors. For example, such embodiments may enable activation of subsets of the fluid injectors that are capable of injecting air and/or oxygen into the combustion zone and deactivation of subsets of fluid injectors that are less suitable for injecting fluid into the combustion zone. As such, embodiments of the biomass gasifier configurations illustrated and described herein may render a single biomass gasifier suitable for use with a variety of types of feedstock, which may be associated with different combustion and pyrolysis zone lengths. However, it should be noted that the illustrated configurations of the controllable fluid injectors are merely exemplary and are not intended to constrain or limit forms which the fluid injectors may take; other sizes, shapes, and configurations are also within the scope of the disclosed fluid injectors.

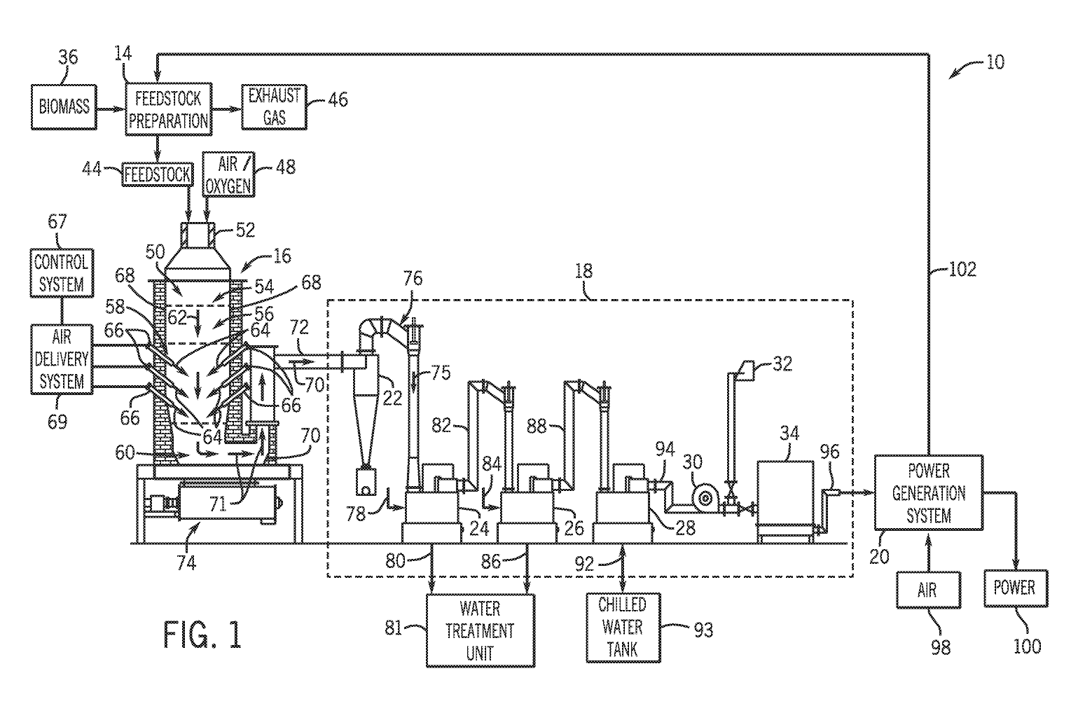

Turning now to the drawings, FIG. 1 illustrates a biomass gasification system 10 that is capable of thermally converting biomass into a more useful gaseous form of fuel (i.e., a fuel form that can be economically utilized with high energy recovery levels) and, subsequently, to clean and cool the gaseous fuel produced via the thermal conversion process. To that end, the illustrated biomass gasification system 10 includes a feedstock preparation unit 14, a biomass gasifier 16, a cleaning and cooling subsystem 18, and a power generation system 20. The cleaning and cooling subsystem 18 includes a cyclone 22, a first scrubber 24, a second scrubber 26, a third scrubber 28, a blower 30, a flare 32, and a filter unit 34. Various conduits are provided that couple these components of the biomass gasification system 10 together, thereby enabling fluid flow between the components, as described in detail below.

During operation of the biomass gasification system 10, biomass 36, is utilized as a natural energy source to generate a more readily usable fuel form, such as producer gas. To that end, the biomass 36 may take the form of any natural or organic material having a molar energy content. For example, the biomass 36 may include one or more of alfalfa straw, bean straw, barley straw, coconut shell, coconut husks, corn cobs, corn fodder, cotton stalks, peach pits, peat, prune pits, rice hulls, safflower, sugarcane, walnut shell, what straw, wood blocks, wood chips, or any other suitable organic feed material.

During operation, the biomass 36 is introduced into the biomass gasifier 16 through the feedstock preparation unit 14, where the biomass 36 may be appropriately processed as desired. Depending on the form of the incoming biomass 36, the feedstock preparation unit 14 may resize or reshape the biomass 36, for example, by chopping, milling, shredding, pulverizing, briquetting, or palletizing the biomass 36. In some embodiments, the feedstock preparation unit 14 may reduce the biomass 36 via densification to a uniformly dimensioned fungible fuel that is sized and shaped to maximize the efficiency of the gasifier 16. In other embodiments, the feedstock preparation unit 14 may receive the biomass 36 as a uniform fuel source and may further process the fuel to customize the processed feedstock 44 for compatibility with the gasifier 16 (e.g., by reducing or increasing moisture content). In instances in which the biomass 36 is partially or completely dried, the feedstock preparation unit 14 may emit a dryer exhaust 46 as part of the drying process.

Once prepared, the processed feedstock 44 and air or oxygen 48 are input into a biomass gasification chamber 50 of the gasifier 16 via inlet 52. In the chamber 50, the biomass-derived feedstock 44 is gasified with the air or oxygen 48 to generate a producer gas with varying concentrations of gases such as carbon monoxide, carbon dioxide, hydrogen, methane, and nitrogen. In particular, the producer gas may be generated by partially combusting the biomass-derived feedstock 44 at an elevated temperature (e.g., approximately 1000° C.). That is, gasification in the biomass gasifier 16 is performed with a surplus of feedstock 44 such that the feedstock 44 is incompletely combusted. The foregoing feature may offer advantages over complete combustion processes by forcing the generation of desirable partial combustion products (e.g., carbon monoxide and hydrogen) while substantially reducing or eliminating the generation of undesirable full combustion byproducts (e.g., nitrogen, water vapor, surplus of oxygen). These desirable partial combustion products, as well as less desirable tar and dust, are produced via reaction of carbon dioxide and water vapor through a layer of heated feedstock-derived charcoal. Therefore, as described in detail below, the gasifier 16 is operated to reduce the biomass-derived feedstock 44 to charcoal and, subsequently, to convert the charcoal to produce carbon monoxide and hydrogen, which, due to their energy rich nature, may be further converted to useful fuel sources such as methanol, ammonia, and diesel fuel via known catalytic processes.

It should be noted that the biomass-derived feedstock 44 may be converted to these higher fuel sources in a variety of suitable types of biomass gasifiers. Specifically, in the embodiments illustrated herein, the thermal conversion process is performed in a downdraft style gasifier, but the illustrated gasifier 16 is not intended to constrain or limit other forms the gasifiers may take during implementation. For example, embodiments of the present invention are compatible with various types of gasifiers, such as downdraft style gasifiers, updraft style gasifiers, crossdraft gasifiers, and so forth. As appreciated by one skilled in the art, the gasifier type chosen for a given gasification system may be dictated by features of the biomass in its final fuel form, such as its size, moisture content, and ash content. For example, in embodiments in which the feedstock 44 may include substantial amounts of tar or dust, a downdraft gasifier may be chosen due to its relative insensitivity to the dust and tar content of the fuel as compared to updraft or crossdraft systems.

Turning now to the operation of the illustrated gasifier 16, the chamber 50 includes a drying zone 54, a pyrolysis zone 56, a combustion zone 58, and a reduction zone 60. It should be noted that the zones are shown as distinct areas of the chamber 50 merely for explanatory purposes but, as appreciated by one skilled in the art, the operational zones would likely exist on a continuum in which the occurring thermal and chemical reactions of one zone often mix with those of the adjacent zones. Further, depending on operational factors, such as the type of biomass 36 being utilized, the respective lengths of each of the zones may differ. As described below, features of the presently disclosed embodiments may render a single gasifier 16 suitable for use with a variety of types of biomass 36 utilized as the feed because air and/or oxygen may be selectively injected into the gasification chamber 50 at locations suitable for the given type of biomass 36 being utilized.

After the biomass-derived feedstock 44 enters the drying zone 54, the moisture content of the feedstock 44 may be reduced from an elevated level (e.g., 10-30%) to a desired level (e.g., 6-10%). In addition to moisture removal in the drying zone 54, the feedstock 44 may also be subjected to reductions in organic acid content. As the dried feedstock 44 flows downstream through the gasifier 16 in the direction indicated by arrows 62 to the pyrolysis zone 56, the feedstock 44 is thermally decomposed at a temperature (e.g., approximately 280-500° C.) that is generally lower than the gasification temperature, typically producing substantial amounts of tar and gases, such as carbon dioxide. The relative amounts of charcoal, tar, and chemicals produced in the pyrolysis zone 56 may depend on the operating conditions within the gasifier 16 (e.g., the temperature at which the pyrolysis occurs) as well as the chemical composition of the feedstock 44. Nevertheless, a condensable hydrocarbon is produced in the relatively low temperature pyrolysis zone 56 regardless of the type of feedstock 44 utilized.

When the decomposed feedstock 44 reaches the combustion zone 58, additional air and/or oxygen 64 is injected into the biomass gasification chamber 50 via a plurality of fluid injectors 66 disposed along a length of a pair of side walls 68 of the biomass gasifier 16. That is, the plurality of fluid injectors 66 are disposed at a variety of locations along the side walls 68, thus enabling the controlled injection of air at a variety of lengthwise locations along the length of the gasification chamber 50. In the disclosed embodiments, the fluid injectors 66 are described as injecting air into the gasification chamber 50. However, it should be noted that the fluid injectors 66 may be adapted to inject air, oxygen, or a combination of air or oxygen with any other suitable gas into the gasification chamber 50. For example, in many embodiments, the air 64 may include inert gases, such as argon and nitrogen, in addition to oxygen or water vapors.

In the illustrated embodiment, a control system 67 controls the supply of air to the plurality of fluid injectors 66 via an air delivery system 69. The control system 67 is capable of exhibiting independent control over the air supply to each of the fluid injectors 66 to control the location or locations along the length of the gasifier 16 at which the air 64 is injected into the gasification chamber 50. For example, the control system 67 may concurrently activate the supply of air from the air delivery system 69 to the fluid injectors 66 in the desired locations along the length of the gasifier 16, and deactivate the supply of air from the air delivery system 69 to the fluid injectors in less desirable locations. In this way, the control system 67 may enable injection of air at selected lengthwise locations along the side walls 68 of the gasifier 16. The foregoing feature may render the biomass gasifier 16 suitable for use in a variety of applications that would typically require separate gasifiers. For example, a gasifier designed for gasification of a sawdust feed may have a substantially longer pyrolysis zone 56 than a gasifier designed for gasification of a wood chip feed. Therefore, the optimal lengthwise location of the fluid injectors along the side walls for the sawdust gasifier would typically be located below the optimal location of the fluid injectors for the wood chip gasifier since it is desirable for the air to be injected into the combustion zone. However, since the plurality of fluid injectors 66 in presently disclosed embodiments are controllable, a suitable subset of the fluid injectors 66 may be activated for any given application depending, for example, on the type of biomass feed being utilized, thus reducing or eliminating the need for multiple gasifiers.

In the illustrated embodiment, in the combustion zone 58, a primary reaction between the carbonized fuel produced in the pyrolysis zone 56 and the injected air produces carbon dioxide in a substantially exothermic reaction (i.e., C+O₂->CO₂). That is, the carbon content of the produced charcoal is partially combusted with oxygen supplied by the fluid injectors 66 to yield carbon dioxide and heat. Concurrently, a secondary reaction takes place between the hydrogen in the fuel and the oxygen in the injected air 64, thereby producing steam (i.e., 2H₂+O₂->2H₂O) in an endothermic reaction that utilizes a portion of the heat produced in the primary reaction. The heat produced in the primary reaction is substantially greater than the heat absorbed in the secondary reaction, thus rendering the overall process occurring in the combustion zone 58 exothermic. As previously mentioned, the overall combustion process occurring in the combustion zone 58 is incomplete and is designed to occur with a surplus of fuel.

The products of this partial combustion (i.e., carbon dioxide, steam, and the uncombusted, partially decomposed pyrolysis products) are exposed to a charcoal bed at an elevated temperature, sparking a series of high temperature chemical reactions in the reduction zone 60. The predominant heat reactions occurring in the reduction zone 60 include a Boudouard reaction (i.e., C+CO₂<->2CO), which is forced to favor the substantially endothermic formation of carbon monoxide due to the high temperatures (e.g., approximately 800-1000° C.) in the reduction zone 60, and a Water Gas reaction (i.e., C+H₂O->CO+H₂), which is also substantially endothermic. Together, these endothermic reduction reactions lower the temperature of the gas flowing through the reduction zone 60. However, slightly exothermic reactions, such as the production of methane from carbon and hydrogen (i.e., C+2H₂->CH₄) also occur in the reduction zone. Still further, operational conditions may be chosen such that additional desired reactions also take place in the reduction zone 60. For example, a Water Shift reaction (i.e., CO₂+H₂->CO+H₂O) may be catalyzed to achieve a desired hydrogen content of the producer gas and, more specifically, to adjust the hydrogen to carbon monoxide (H/CO) ratio of the producer gas to an appropriate level for the downstream application. Accordingly, at an outlet 70 of the gasifier 16, a producer gas indicated by arrow 71 is routed to conduit 72 for transmission to the cleaning and cooling subsystem 18.

As appreciated by one skilled in the art, the composition of the producer gas 71 is subject to considerable variations and depends on factors such as the biomass type, operational parameters of the gasifier 16, and so forth, and may include varying concentrations of gases such as carbon monoxide, hydrogen, methane, carbon dioxide, and nitrogen. For example, in instances in which air instead of oxygen is injected via fluid injectors 66, the producer gas 71 may include a greater volumetric concentration of nitrogen. Still further, the temperature of the producer gas 71 at the outlet 70 of the gasifier 16 may be between approximately 300° C. and approximately 400° C. However, the producer gas 71 is subject to considerable variations in temperature based on biomass type and operational conditions. For example, in gasifiers 16 in which the operational flow velocity through the gasifier 16 exceeds the desired air flow rate, the temperature of the producer gas 71 may be higher than desired (e.g., greater than approximately 500° C.).

Concurrent with the flow of producer gas 71 through the outlet 70 of the gasifier 16, hot ash exits the gasifier 16 via an ash extraction system 74. The hot ash may be derived from the mineral content of the fuel that remains in oxidized form after the combustion zone 58. The ash extraction system 74 receives the hot ash generated during the biomass gasification and contains the hot ash for subsequent removal from the biomass gasifier 16. If desired for the given application, one or more heat exchangers may be placed in the ash extraction system 74 to cool the hot ash via convection.

Whereas the hot ash remains in the ash extraction system 74 for removal, the producer gas 71 flows through conduit 72 to the cyclone 22. The cyclone 22 is a dry filter that may be operated to remove dust and other particles from the producer gas 71. For example, the cyclone 22 may be used to filter out particles equal to or greater than approximately 5 micrometers. In some embodiments, approximately 60 to 65 percent of the producer gas 71 may comprise particles greater than 60 micrometers in size; therefore, the cyclone 22 may remove a large number of particles from the producer gas 71.

After filtering in the cyclone 22, the producer gas 71 flows through a conduit 76 to the first scrubber 24 where the filtered producer gas 71 is cleaned, for example, by removing tar and entrained gases, such as hydrogen cyanide. In particular, within the first scrubber 24, fines and tar may be separated from the producer gas 71 with clean water, as indicated by arrow 78, to produce a stream of black water 80 that exits a bottom portion of the first scrubber 24 and is directed to a black water processing system located within a water treatment unit 81. The scrubbed producer gas 71 exits the first scrubber 24 and is transferred to the second scrubber 26 via conduit 82.

In the second scrubber 26, additional fines, tar, and gases may be removed with clean water 84. As before, the fines and tar may be separated from the producer gas to produce a second stream of black water 86 that may exit a bottom portion of the second scrubber 26 and be directed to a black water processing system located within the water treatment unit 81. In some embodiments, the water treatment unit 81 may include a series of flash tanks that subject the black water 80 and 86 to a series of pressure reductions to remove dissolved gases and to separate and/or concentrate the fines. The separated fines may be recycled and used in the feedstock preparation unit 14 to provide additional biomass 36 for the biomass gasifier 16 if desired.

The scrubbed producer gas exiting the second scrubber 26 flows through conduit 88 to the third scrubber 28, which may be a chilled water scrubber. In the third scrubber 28, the producer gas may be cooled with chilled water 92 that flows into the third scrubber 28, exchanges heat with the hot producer gas, and subsequently flows back to a chilled water tank 93 where the water is cooled for recirculation. The cooled producer gas flows through conduit 94 to the blower 30. The blower 30 is operated to pull the producer gas 71 from the biomass gasifier 16 through the gas cleaning and cooling subsystem 18. If desired, an excess portion of the producer gas may be burned by flare 32.

The unburned portion of the producer gas flows from the blower 30 to the filtering unit 34. The filtering unit 34 includes one or more filter elements configured to extract particulates from the producer gas. The cleaned and filtered producer gas is routed from the gas cleaning and cooling subsystem 18 to the power generation system 20, where the producer gas may be utilized to produce power. For example, the power generation system 20 may include a gas engine that combusts the producer gas 71 with air 98 to produce power 100 for a downstream application. For example, the power 100 may be used to directly operate other systems and/or to provide power to a utility grid. During combustion, the gas engine may produce engine exhaust 102, which may be used to dry the biomass 36 in the feedstock preparation unit 14 in some embodiments.

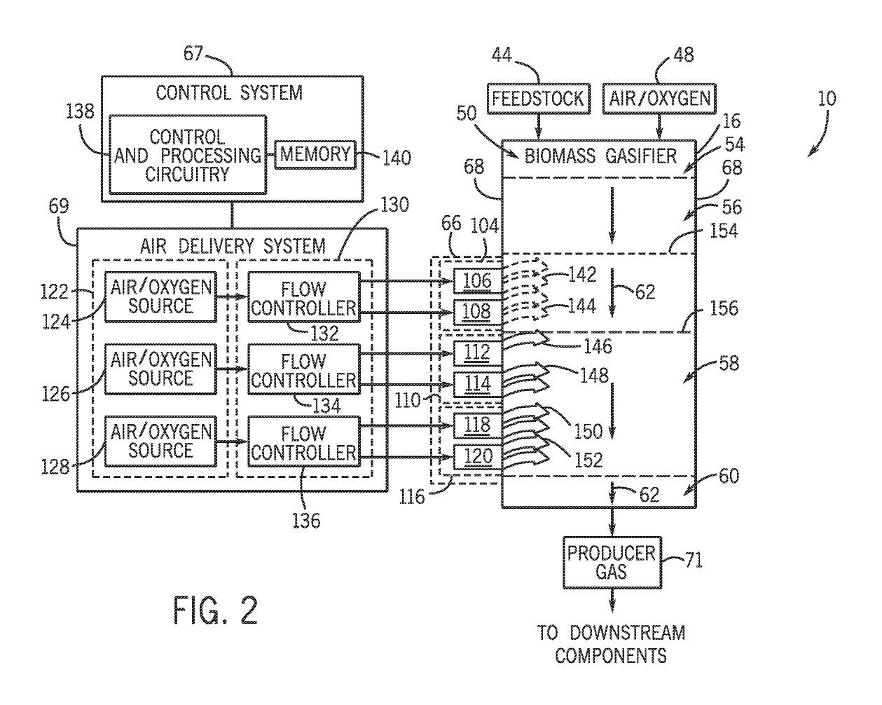

FIG. 2 is a diagram of an embodiment of the biomass gasification system 10 that may be used to generate producer gas 71 in accordance with the presently disclosed embodiments. The biomass gasification system 10 includes the biomass gasifier 16 that converts the biomass feedstock 44 into the producer gas 71 via pyrolytic heating with air or oxygen. To that end, the biomass gasifier 16 includes the plurality of fluid injectors 66 disposed lengthwise along the side walls 68 of the gasification enclosure in a flow direction from the gasifier inlet to the gasifier outlet in the flow direction. In the depicted embodiment, the fluid injectors 66 are shown as perpendicular to the gasifier walls. However, in other embodiments, the fluid injectors 66 may be angled with respect to the walls of the gasifier, for example, as shown in FIG. 1. In the illustrated embodiment, the plurality of fluid injectors 66 includes a first subset 104 of fluid injectors 66 including fluid injectors 106 and 108; a second subset 110 of fluid injectors 66 including fluid injectors 112 and 114; and a third subset 116 of fluid injectors 66 including fluid injectors 118 and 120. However, it should be noted that in certain embodiments, each fluid injector 106, 108, 112, 114, 118, and 120 may represent a single injector or a plurality of injectors distributed about the gasification chamber 50. In addition, in certain embodiments, additional numbers of subsets as well as additional numbers of fluid injectors within a given subset other than the illustrated quantities may be provided. Still further, it should be noted that although in the illustration of FIG. 2, the fluid injectors are shown on a single side of the gasification chamber 50, as would be understood by those skilled in the art, presently disclosed embodiments may include fluid injectors disposed about the circumference of the gasifier.

As described above, air/oxygen is supplied to the plurality of fluid injectors 66 (e.g., 106, 108, 112, 114, 118, and 120) by the air delivery system 69. The illustrated air delivery system 69 includes an air source set 122 including independent air/oxygen sources 124, 126, and 128, and a flow controller set 130 including independent flow controllers 132, 134, and 136. Each of the components of the air delivery system 69 is controlled by the control system 67 that includes control and processing circuitry 138 and memory 140. The memory 140 may include any suitable type of memory, including but not limited to read only memory (ROM), random access memory (RAM), magnetic storage memory, optical storage memory, or a combination thereof.

During operation of the biomass gasification system 10, the control circuitry 138 is capable of independently controlling air/oxygen flow associated with each of the air sources 124, 126, and 128 by independently controlling the flow controllers 132, 134, and 136. For example, in the illustrated embodiment, the air source 124 and the flow controller 132 may be concurrently operated to activate or deactivate the flow of air to the first subset of fluid injectors 104. When the first subset 104 of fluid injectors 104 is activated, the fluid injectors 106 and 108 receive air flowing along an airflow path from the air source 124 and through the flow controller 132, and air 142 and 144 is injected into the gasification chamber 50. Similarly, the airflow path including the air source 126 and the flow controller 134 supplies the fluid injectors 112 and 114 with air 146 and 148 that is injected into the gasification chamber 50. Likewise, air 150 and 152 is supplied to the fluid injectors 118 and 120 from the airflow path that includes the air source 128 and the flow controller 136.

As described in detail above, the biomass feedstock 44 and the air/oxygen 48 are injected into the biomass gasifier 16 and flow in direction 62 through the drying zone 54, the pyrolysis zone 56, the combustion zone 58, and the reduction zone 60 to produce the producer gas 71. As also described above, the plurality of fluid injectors 66 may be independently controlled to optimize the production of the producer gas 71 based on a variety of operational parameters, such as the type of feedstock 44 being utilized in the given process. For example, the plurality of fluid injectors 66 may be independently controlled such that additional air/oxygen is injected only into the approximated combustion zone 58 associated with the given biomass gasification process (i.e., for the particular biomass 36 and feedstock 44). For further example, the plurality of fluid injectors 66 may be independently controlled such that air/oxygen is injected only into a bottom portion of the combustion zone 58 (e.g., an approximate bottom quarter or half of the combustion zone).

For instance, in one embodiment, the feedstock 44 may be wood chips, and the combustion zone 58 may begin at approximately the lengthwise location indicated by dashed line 154. In this embodiment, the first, second, and third subsets of fluid injectors 104, 110, and 116 may all be activated since all the fluid injectors 106, 108, 112, 114, 118, and 120 inject air into the combustion zone 58 that begins at dashed line 154. If air injection is only desired in a bottom portion of the combustion zone 58, however, only the third subset 116 of fluid injectors may be activated for use with the wood chip feed.

In another embodiment, the feedstock 44 may be sawdust, and the combustion zone 58 may begin at approximately the lengthwise location indicated by dashed line 156. In this embodiment, the first subset of fluid injectors 104 may be deactivated since the fluid injectors 106 and 108 are located at a lengthwise location along the side walls 68 that is not suitable for injection of air into the combustion zone 58 that begins at dashed line 156. The second subset 110 and/or the third subset 116 of fluid injectors may then be activated, depending on whether injected air/oxygen is desired throughout the combustion zone 58 or only in a bottom portion thereof. It should be noted that the illustrated and described embodiments are merely exemplary, and in additional embodiments, any subset of the plurality of fluid injectors 66 may be independently controlled to inject air/oxygen at a desired lengthwise position along the side walls 68 of the biomass gasifier 16.

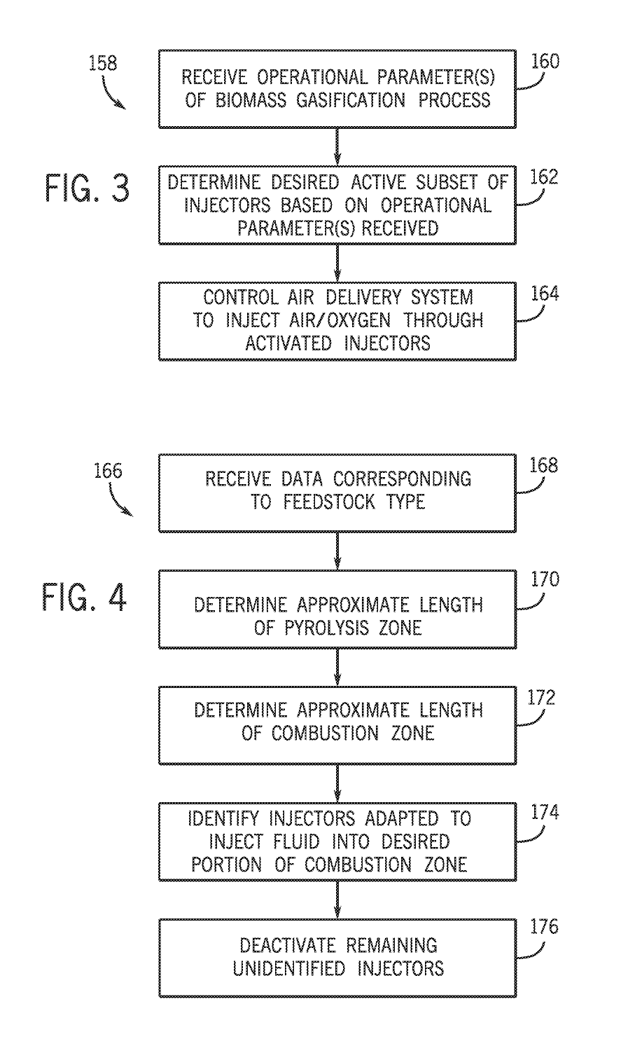

FIG. 3 illustrates an embodiment of a method 158 that may be implemented by the control system 67 to selectively activate the plurality of controllable fluid injectors 66 based on an operational parameter of a biomass gasification process. The method 158 includes receiving an operational parameter of the biomass gasification process (block 160) and, based on the received parameter, determining desired subsets of the fluid injectors 66 for activation (block 162). For example, as previously described, the operational parameter may be the type of biomass 36 and feedstock 44 being utilized, and the activated subsets of fluid injectors 66 may be the fluid injectors capable of injecting air and/or oxygen into the approximate combustion zone 58 associated with the type of biomass 36 and feedstock 44. Further, the method 158 includes controlling the air delivery system 69 to inject air and/or oxygen into the gasification chamber 50 through the activated fluid injectors 66 (block 164).

FIG. 4 illustrates an embodiment of a method 166 that the control system 67 may utilize when the operational parameter is the type of biomass 36 and feedstock 44. The method 166 includes receiving data corresponding to the biomass and feedstock type (block 168) and determining an approximate length of the pyrolysis zone corresponding to that feedstock type (block 170). The pyrolysis zone length may be utilized to determine the approximate length of the combustion zone 58 (block 172) and its corresponding location along the side walls 68 of the biomass gasifier 16. The lengths of the pyrolysis and combustion zones may be determined, for example, based on user input, prior knowledge of previous gasification processes, automatically computed by the control system, based on a mathematical model, and so forth. Subsequently, the fluid injectors 66 capable of injecting air/oxygen into the combustion zone 58 (or the desired portion of the combustion zone 58) may be identified (block 174), and the unidentified fluid injectors 66 may be deactivated (block 176).

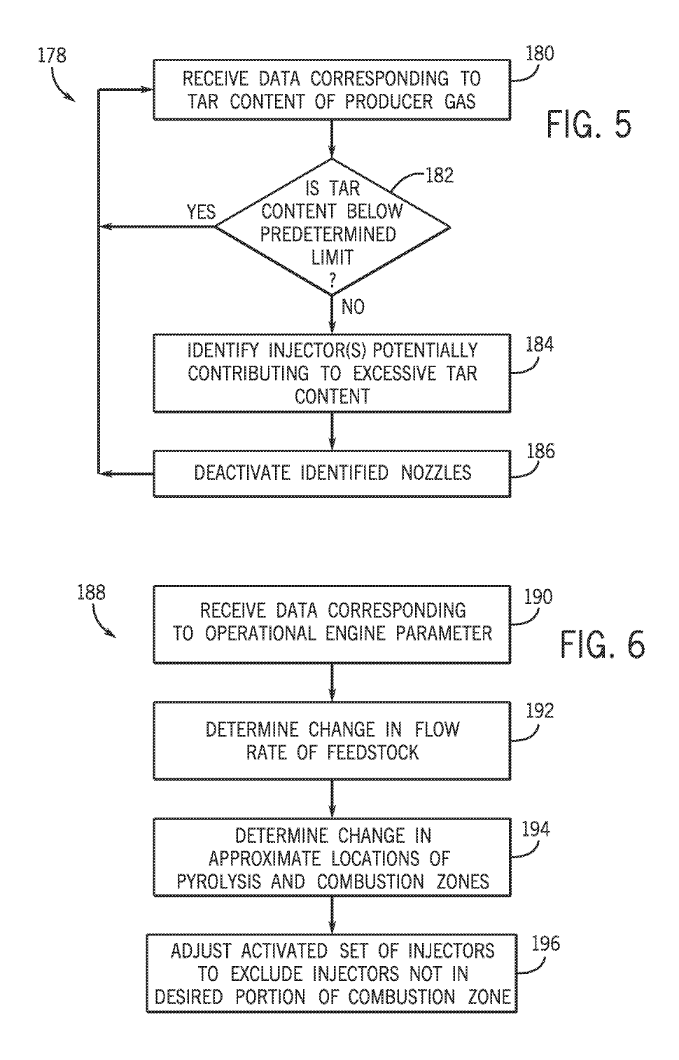

FIG. 5 illustrates an embodiment of a method 178 that may be implemented by the control system 67 to control the fluid injectors 66 based on a detected tar content of the producer gas 71. Specifically, the method 178 includes receiving data corresponding to the tar content of the producer gas 71 (block 180), which may be acquired, for example, via a sensor located at the outlet 70 of the biomass gasifier 16. The method 178 proceeds by determining whether the tar content present in the producer gas 71 is below a predetermined limit (block 182). For example, the predetermined limit may be a limiting percentage of the producer gas 71 by weight or volume that is allowed to be tar. If the tar content is below the predetermined limit, the tar content of the producer gas 71 is monitored. However, if the tar content exceeds the predetermined limit, the control system 67 identifies one or more fluid injectors 66 that may be contributing to the excessive tar content in the producer gas 71 (block 184) and deactivates the identified fluid injectors 66 (block 186). Data corresponding to the tar content of the producer gas 71 may then be received (block 180), and the process may be repeated until the tar content is below the predetermined limit.

FIG. 6 illustrates an embodiment of a method 188 that the control system 67 may implement to control the plurality of fluid injectors 66 when the received operational parameter is a parameter of a component of the power generation system 20. For example, in certain embodiments, the control system 67 may receive data corresponding to an operational parameter of an engine (block 190) and exhibit selective control over the plurality of fluid injectors 66 based on the engine parameter. For example, in some embodiments, as the power output of the engine changes, the air intake into the biomass gasifier 16 may also change, and the ash removal grate speed may be adjusted to maintain a desired air to fuel ratio at the inlet of the biomass gasifier 16. As such, the residence time of the feedstock 44 flowing through the biomass gasifier 16 changes, and the change in the flow rate of the feedstock through the gasification chamber 50 is determined by the control system (block 192).

The determined change in the flow rate of the feedstock 44 through the biomass gasifier 16 may be utilized by the control circuitry 138 to determine a corresponding change in the approximate locations of the pyrolysis and combustion zones 56 and 58 in the gasification chamber 50 (block 194). Here again, the control system 67 may then selectively control the plurality of fluid injectors 66 to ensure that air/oxygen is being injected into the gasification chamber at the desired locations along the length of the side walls 68 of the gasifier 16. For example, in the illustrated method 188, the control system 67 adjusts the activated subsets of fluid injectors 66 to exclude the injectors that are not capable of injecting air/oxygen into the desired portion of the combustion zone 58 (block 196).

In the illustrated methods, the control system 67 controls the plurality of fluid injectors 66 based on parameters such as the tar content of the producer gas 71, the type of biomass 36 or feedstock 44, or an operational parameter of an engine of the power generation system 20. However, it should be noted that these embodiments are merely exemplary, and the control system logic employed in a particular biomass gasification system 10 may be subject to considerable implementation-specific variations. That is, the control system 67 may utilize various operational or process specific parameters to independently control the plurality of fluid injectors 66, not limited to the specific parameters described herein.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A biomass gasification system, comprising:
a reactor configured to gasify a biomass feedstock to thermally convert the biomass feedstock into producer gas, wherein the reactor comprises:
an enclosure disposed about a biomass gasification chamber, wherein the enclosure comprises an inlet, an outlet, and side walls disposed between the inlet and the outlet; and
a plurality of fluid injectors disposed along a length of the side walls and configured to inject fluid into the gasification chamber; and
a control system communicatively coupled to the plurality of fluid injectors and configured to independently control each fluid injector of the plurality of fluid injectors to independently control a flow of fluid through each fluid injector.

2. The biomass gasification system of claim 1, wherein the fluid comprises air, oxygen, or a combination thereof.

3. The biomass gasification system of claim 1, wherein the plurality of fluid injectors comprises a plurality of subsets of fluid injectors, and wherein each fluid injector within a subset of fluid injectors is configured to be activated and deactivated concurrent with the activation and deactivation of each other fluid injector in the subset.

4. The biomass gasification system of claim 1, wherein the control system is configured to selectively control each fluid injector based on an operational parameter of the reactor.

5. The biomass gasification system of claim 4, wherein the operational parameter comprises a type of the biomass feedstock, an approximate length of a pyrolysis zone of the gasification chamber, an approximate length of a combustion zone of the biomass gasification chamber, an operational parameter of an engine that receives the producer gas from the reactor, a tar content of the producer gas, or a combination thereof.

6. The biomass gasification system of claim 1, comprising an air delivery system configured to supply the plurality of fluid injectors with air, oxygen, or a combination thereof.

7. The biomass gasification system of claim 6, wherein the air delivery system comprises an air source, an oxygen source, a flow controller, or a combination thereof.

8. The biomass gasification system of claim 1, comprising a cyclone configured to separate particulates from the producer gas.

9. The biomass gasification system of claim 1, comprising a scrubber system configured to clean the producer gas.

10. The biomass gasification system of claim 1, comprising a power generation system configured to receive the producer gas and to utilize the producer gas to generate at least one of methanol, ammonia, and diesel fuel.

11. A method, comprising:
receiving data corresponding to an operational parameter of a biomass gasification process or a power generation system that receives producer gas from the biomass gasification process;
activating, based on the received data, a subset of a plurality of air and/or oxygen injectors disposed along a length of a biomass gasification chamber; and
controlling an air delivery system to supply the activated subset of the plurality of air and/or oxygen injectors with air and/or oxygen for injection into the biomass gasification chamber.

12. The method of claim 11, wherein the operational parameter comprises a type of biomass feedstock utilized in the biomass gasification process.

13. The method of claim 11, wherein the operational parameter comprises an approximate length of a pyrolysis zone of the biomass gasification chamber.

14. The method of claim 11, wherein the operational parameter comprises an approximate length of a combustion zone of the biomass gasification chamber.

15. The method of claim 11, wherein the operational parameter comprises an operational parameter of an engine.

16. The method of claim 11, wherein the operational parameter comprises a tar content of a producer gas produced in the biomass gasification process.

17. The method of claim 11, wherein the activated subset of the plurality of air and/or oxygen injectors is capable of injecting air and/or oxygen into a combustion zone of the biomass gasification chamber.

18. The method of claim 11, wherein controlling the air delivery system comprises activating an air and/or oxygen source and a flow controller corresponding to each of the air and/or oxygen injectors included in the subset.

19. The method of claim 11, comprising deactivating, based on the received data, a second subset of the plurality of air and/or oxygen injectors disposed along the length of the biomass gasification chamber.

20. The method of claim 19, comprising controlling the air delivery system to deactivate an air and/or oxygen source and a flow controller corresponding to each of the air and/or oxygen injectors included in the second subset.

21. A biomass gasification system, comprising:
a reactor configured to gasify a biomass feedstock in a biomass gasification chamber to thermally convert the biomass feedstock into producer gas, wherein a plurality of nozzles are disposed along a length of the reactor and configured to inject air and/or oxygen into the biomass gasification chamber; and
a control system configured to determine an approximate location of a combustion zone in the gasification chamber and to selectively deactivate each nozzle of the plurality of nozzles not capable of injecting air and/or oxygen into the combustion zone.
